(19) United States
(12) Patent Application Publication
(10) Pub. No.: US 2012/0186589 A1
(43) Pub. Date: Jul. 26, 2012

(54) APPARATUS AND METHOD FOR TREATMENT OF SLEEP APNEA AND SNORING

(76) Inventor: Pankaj P. Singh, Brookville, NY (US)

(21) Appl. No.: 13/014,646

(22) Filed: Jan. 26, 2011

(51) Int. Cl. A61F 5/56; A61C 9/00

(52) U.S. Cl. 128/848; 433/214

(57) ABSTRACT

Methods and apparatus for treating snoring and sleep apnea may include an oral appliance based on an extensive, three-dimensional radiological and clinical examination. The oral appliance may position the patient's lower jaw to a position that may prevent or eliminate sleep apnea. Several parameters, often over 40 parameters, may be examined to determine the proper placement and orientation of the jaw, via the oral appliance. The oral appliance may have a soft insert on the inside for cushioning the patient's teeth while the outside of the oral appliance may be hard, so as to maintain airflow through the oral appliance. The oral appliance may be a “full coverage” piece, fitting over 70 percent, typically over 80 percent, of the patient's teeth.

### BACKGROUND OF THE INVENTION

The present invention relates to methods and apparatus for the treatment of snoring and sleep apnea and, more particularly, to an oral appliance and methods for fitting an oral appliance for the treatment of sleep apnea.

Sleep apnea is a treatable disorder in which a person literally stops breathing hundreds of times at night, for at least 10 seconds at a time, during sleep. The soft tissue in the rear of the throat collapses and closes the airway, which causes oxygen levels to drop and carbon dioxide levels to rise. Sleep apnea affects men, women and children of all ages and any weight. Studies have shown 1 of 3 adults suffer from sleep related breathing disorders and that 3% of children have hyperactivity and difficulty in school due to sleep apnea.

Sleep apnea can trigger or exacerbate many health problems, such as high blood pressure, increased risk for heart attacks and strokes, memory loss, diabetes, impotence, weight gain, depression, impaired mental functioning, delayed reaction times and high pressures in the lung arteries. Sleep deprivation affects an individual's alertness and decision-making ability. Studies have shown that sleep apnea is responsible for 1 in 3 fatalities in motor vehicle accidents and causes 1,400 tragic fatalities in America each year. Recent studies have shown that Snoring causes Carotid Atherosclerosis by causing plaque accumulation along the inside lining of the Carotid Arteries.

Treatment of sleep apnea often focuses on a continuous positive airway pressure (CPAP) apparatus. The CPAP apparatus forces the patient's airway open during sleep by means of a flow of pressurized air into the throat. The patient typically wears a plastic facial mask, which is connected by a flexible tube to a small bedside CPAP machine air compressor. The CPAP machine generates the required air pressure to keep the patient's airways open during sleep. However, some patients cannot tolerate CPAP and must find other ways to deal with or treat their sleep apnea.

As can be seen, there is a need for other methods and apparatus for treating sleep apnea.

### SUMMARY OF THE INVENTION

In one aspect of the present invention, a oral appliance comprises an upper teeth fitting section; a lower teeth fitting section connected to the upper teeth fitting section with a plurality of connecting bridges, the plurality of connecting bridges fixing the position of the lower teeth fitting section relative to the upper teeth fitting section; and airway passages formed between the connecting bridges, wherein the lower teeth fitting section is adapted to position a user's lower jaw to reduce or prevent sleep apnea, wherein the position is determined by a sleep disorder examination of the user.

In another aspect of the present invention, a method for fitting a patient with an oral appliance comprises taking an impression of the user's upper and lower teeth; molding an upper teeth fitting section to correspond to the user's upper teeth; molding a lower teeth fitting section to correspond to the user's lower teeth; performing a sleep disorder examination of the patient to determine a suitable position of the user's mandible relative to the user's maxilla; and rigidly connecting the upper and lower teeth fitting sections in accordance with the results of the sleep disorder examination.

In a further aspect of the present invention, a method for the treatment of sleep apnea comprises fitting a patient in need thereof with an oral appliance, the oral appliance fixing the position of the mandible relative to the maxilla, the oral appliance comprises an upper teeth fitting section; a lower teeth fitting section connected to the upper teeth fitting section with a plurality of connecting bridges, the plurality of connecting bridges fixing the position of the lower teeth fitting section relative to the upper teeth fitting section; and airway passages formed between the connecting bridges, wherein the lower teeth fitting section is adapted to position the patient's lower jaw to reduce or prevent sleep apnea, wherein the position is determined by a sleep disorder examination of the patient.

These and other features, aspects and advantages of the present invention will become better understood with reference to the following drawings, description and claims.

### BRIEF DESCRIPTION OF THE DRAWINGS

FIG. 1 is a perspective view of an oral appliance according to an exemplary embodiment of the present invention;

FIG. 2 is a left side perspective view of the oral appliance of FIG. 1;

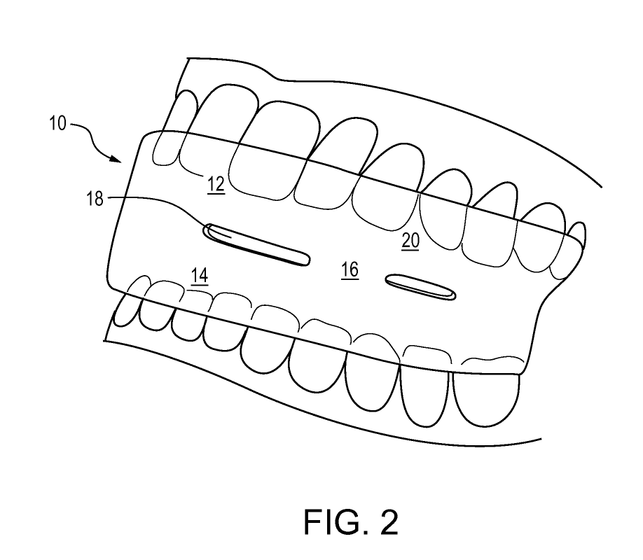

FIG. 3 is a right side perspective view of the oral appliance of FIG. 1;

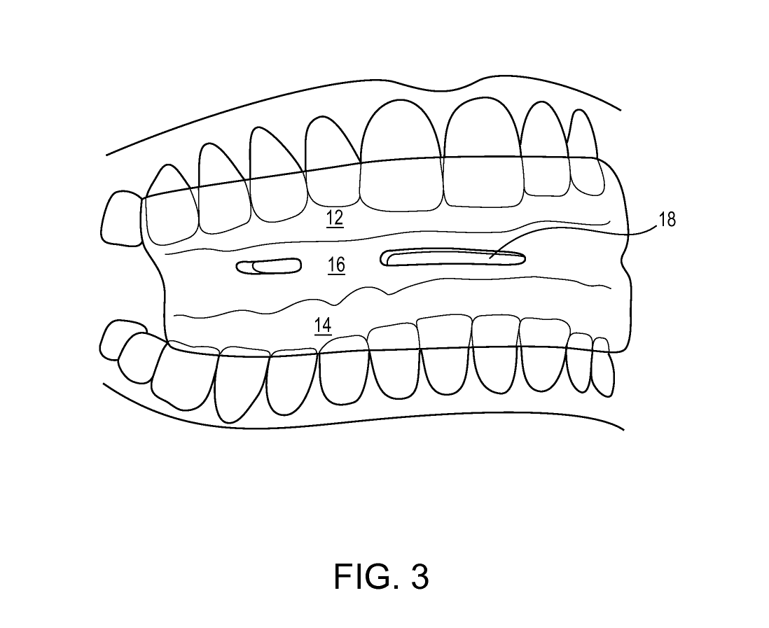

FIG. 4 is an axial x-ray view of a patient after placement of the oral appliance of FIG. 1;

FIG. 5 is an axial x-ray view of the patient of FIG. 4, without the oral appliance in place;

FIG. 6 is a lateral x-ray view of the patient of FIG. 4 after placement of the oral appliance of FIG. 1; and

FIG. 7 is a lateral x-ray view of the patient of FIG. 4, without the oral appliance in place.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides methods and apparatus for treating sleep apnea. The methodology may include an extensive, three-dimensional radiological and clinical examination. The apparatus may include an oral appliance that may position the patient's lower jaw to a position that may prevent or eliminate sleep apnea. Several parameters, often over 40 parameters, may be examined to determine the proper placement and orientation of the jaw, via the oral appliance. The oral appliance may have a soft insert on the inside for cushioning the patient's teeth while the outside of the oral appliance may be hard, so as to maintain airflow through the oral appliance. The oral appliance may be a “full coverage” piece, fitting over 70 percent, typically over 80 percent, of the patient's teeth.

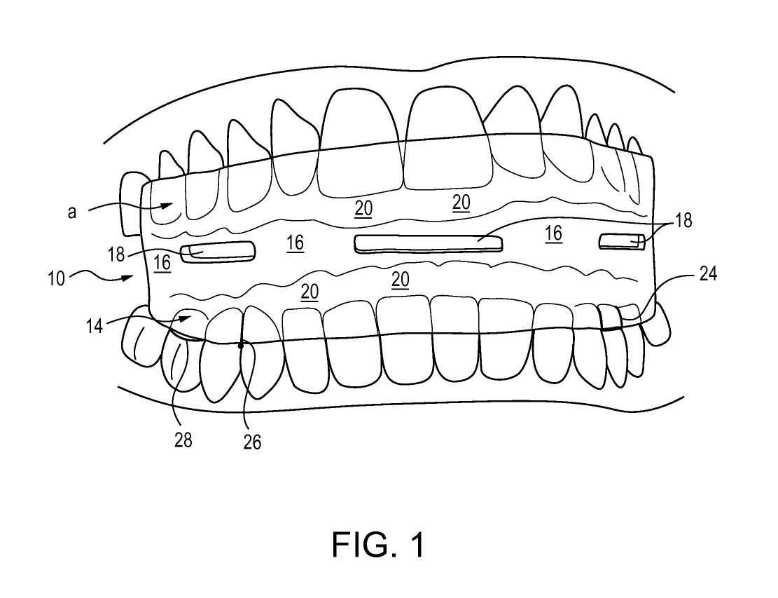

Referring to FIG. 1, an oral appliance 10 (also referred to as appliance 10) may include an upper teeth fitting portion 12 and a lower teeth fitting portion. The upper teeth fitting portion 12 may be adapted to receive substantially all of the patient's teeth therewithin. Similarly, the lower teeth fitting portion 14 may be adapted to receive substantially all of the patient's lower teeth therewithin.

Typically over 70%, often over 80%, of the patient's teeth fit into the oral appliance 10. The appliance 10 may include a plurality of connecting braces 16 between the upper teeth fitting portion 12 and the lower teeth fitting portion 14, fixing the position of the upper teeth fitting portion 12 relative to the lower teeth fitting portion 14. A plurality of breathing openings 18 may be formed in between connecting braces 16. The appliance 10 may be considered a mandible repositioning device, as it may fix the patient's mandible at a positioned as determined by the below described methods.

The appliance 10 may be made of a rigid material, such as, for example, a polymeric material such as an epoxy, a polymer-like self-curing acrylic, a thermoplastic polymer material or the like. The appliance 10 may be made from an impression of the patient's teeth. A soft insert 20 made from, for example a soft, cold or thermal cured polymer, may be positioned inside the upper teeth fitting portion 12 and the lower teeth fitting portion 14. The soft insert may provide a cushion for the teeth inside the appliance 10.

The appliance 10 may include one or more clasps for connecting the appliance 10 to a user's teeth. In some embodiments, the clasps may be adapted to connect one or more of the user's lower teeth to the lower teeth fitting portion 14. Various styles of clasps may be used, as are known in the art. The clasps may be embedded inside the lower teeth fitting portion 14, go around the molar and/or premolar and/or canine teeth for aid in retention of the appliance 10. The clasps can be, for example, circumferential 28 (around the teeth) or ball 26 (goes between the teeth in the embrasure space) or Adams 24 (goes over the tooth and onto the facial surface of the teeth).

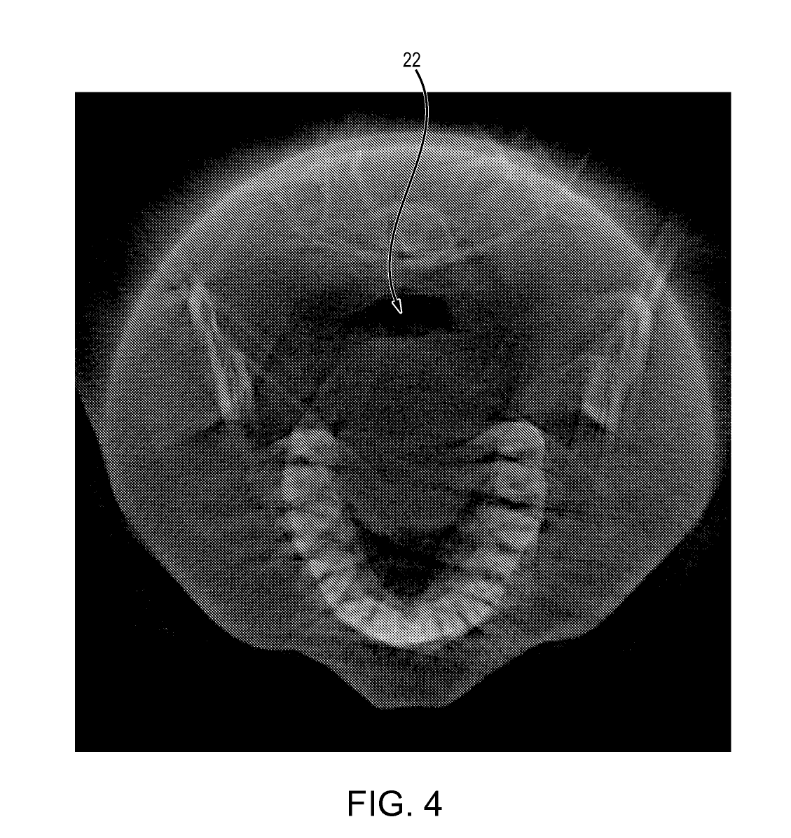

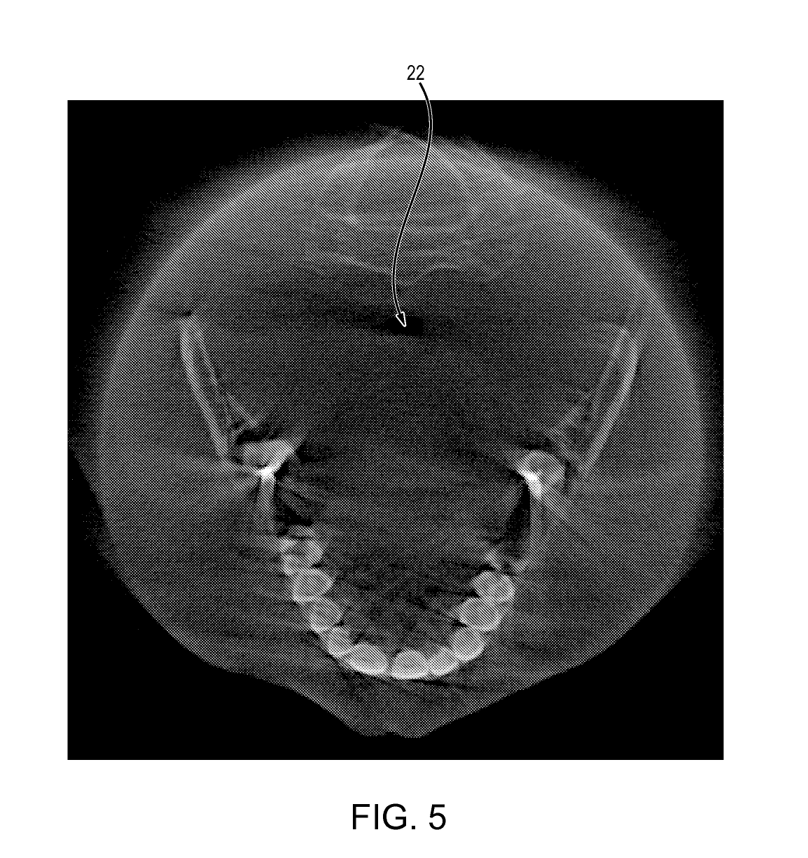

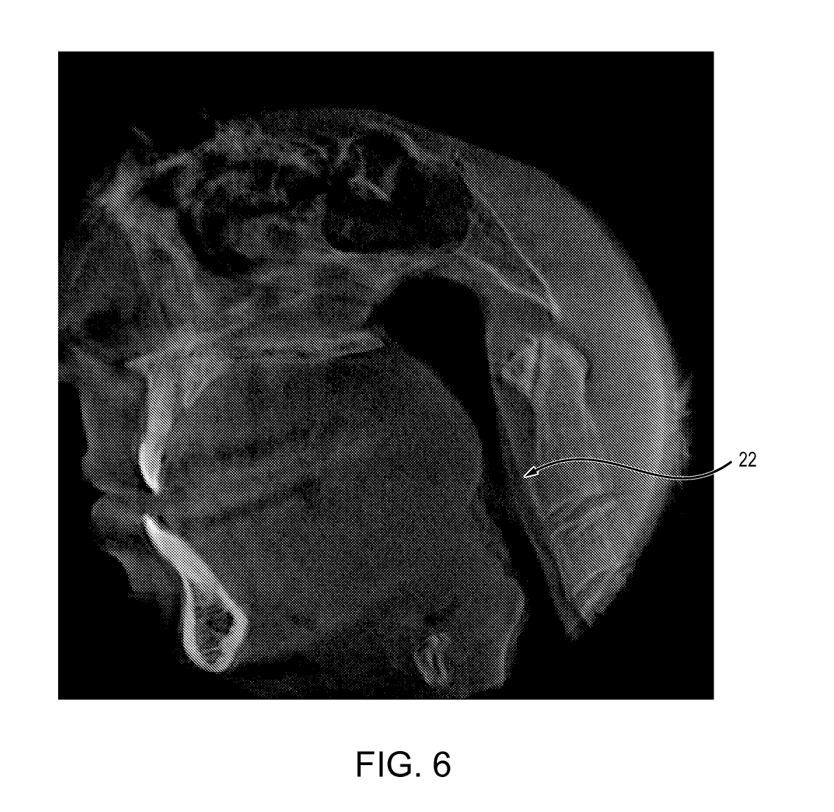

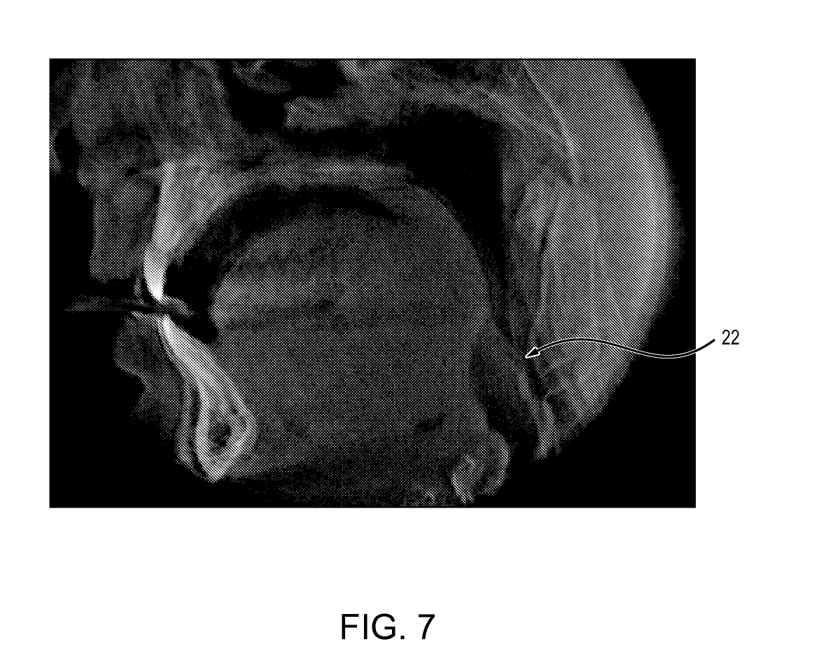

Referring to FIGS. 4 and 5, there is shown an axial x-ray of a patient after and before, respectively, placement of the oral appliance 10. As can be seen from the figures, an airway opening 22 is larger when the appliance 10 is used. Similarly, FIGS. 6 and 7 show a frontal x-ray of a patient after and before, respectively, placement of the oral appliance 10. The airway opening 22 in these figures is more open with the appliance 10 is in place in the patient's mouth.

The placement of the upper teeth fitting portion 12 with respect to the lower teeth fitting portion 14 may be determined through an extensive three-dimensional radiological and clinical examination. Various parameters are described in detail below.

A method for determining whether a patient may be a candidate for the oral appliance may include giving the patient a sleep disorder examination. The examination may include obtaining a base-line set of vitals (blood pressure, pulse, oxygen saturation, neck measurement, and the like). An airway evaluation may include characterizing the tongue, determining the level of the tongue, characterizing the patient's swallow, classifying the patient's mallampati, classifying the patient's tonsils, and classifying the patient's adenoids, uvula, soft palate, gag reflex, nasal passages, turbinates, maxilla and mandible. A dental screening may include an examination of the patient's teeth, noting any disease, tooth loss, fractures, prosthesis, restorations and the like. The dental screening may also include a determination of dental relationship (class and division) and any tooth contact prior to the oral appliance therapy.

The sleep disorder examination may also include an evaluation of the patient's facial muscles, noting any tenderness upon palpation. Joint sounds, such as crepitus or clicking, may be noted and the patient's jaw range of motion may be determined. The patient may also undergo a craniomandibular screening.

The sleep disorder examination may further include diagnostic testing. The diagnostic tests may include a cephalogram, CT scan, frontal skull (PA) imagine, a full mouth series, lateral skull imagine, MRI, overnight pulse oximetry, panores, pharyngometer, rhinometer, TMJ tomography and transcranial imaging. A sleep study, either monitored or unattended, may be part of the diagnostic testing of the sleep disorder examination.

The apparatus and method of the present invention may have several advantages. For example, the method may be conservative, effective, lightweight and easy to adjust. The apparatus may be similar to a conventional orthodontic appliance and may be a cost and time effective alternative to CPAP and/or surgery. The apparatus may be modeled to each patient's jaw to an exact position, which is individualized for each patient.

For patient's who snore with mild sleep apnea, mild to moderate sleep apnea, have sleep apnea but cannot tolerate CPAP and refuse surgery, or have found both the CPAP and surgery to be unsuccessful, a dental sleep appliance, according to the present invention as described above, may provide a conservative and successful alternative for the treatment of sleep apnea.

It should be understood, of course, that the foregoing relates to exemplary embodiments of the invention and that modifications may be made without departing from the spirit and scope of the invention as set forth in the following claims.

What is claimed is:

1. An oral appliance comprising:
an upper teeth fitting section;
a lower teeth fitting section connected to the upper teeth fitting section with a plurality of connecting bridges, the plurality of connecting bridges fixing the position of the lower teeth fitting section relative to the upper teeth fitting section; and
airway passages formed between the connecting bridges, wherein
the lower teeth fitting section is adapted to position a user's lower jaw to reduce or prevent sleep apnea, wherein the position is determined by a sleep disorder examination of the user.

2. The oral appliance of claim 1, wherein the upper and lower teeth fitting sections contain at least 70% of the user's teeth therewithin.

3. The oral appliance of claim 1, further comprising at least one clasp embedded in the lower teeth fitting section, the clasp adapted to connect the appliance to one or more of the user's teeth.

4. The oral appliance of claim 1, wherein a soft material is inserted into the upper and lower teeth fitting sections to cushion the teeth inside the oral appliance when worn by the user.

5. The oral appliance of claim 1, further comprising at least four connecting bridges and at least three airway passages formed between adjacent ones of the connecting bridges.

6. A method for fitting a patient with an oral appliance, the method comprising:
taking an impression of the user's upper and lower teeth;
molding an upper teeth fitting section to correspond to the user's upper teeth;
molding a lower teeth fitting section to correspond to the user's lower teeth;
performing a sleep disorder examination of the patient to determine a suitable position of the user's mandible relative to the user's maxilla; and
rigidly connecting the upper and lower teeth fitting sections in accordance with the results of the sleep disorder examination.

7. The method of claim 6, wherein the suitable position is a position that reduces or eliminated sleep apnea in the user.

8. The method of claim 6, wherein the sleep disorder examination includes three-dimensional radiological and clinical examinations.

9. The method of claim 6, wherein the sleep disorder examination includes taking the user's vital data, an airway evaluation, mallampati classification, tonsil classification, and a dental screening.

10. The method of claim 6, further comprising inserting a soft material is into the upper and lower teeth fitting sections to cushion the teeth inside the oral appliance when worn by the user.

11. A method for the treatment of sleep apnea, the method comprising fitting a patient in need thereof with an oral appliance, the oral appliance fixing the position of the mandible relative to the maxilla, the oral appliance comprising:
an upper teeth fitting section;
a lower teeth fitting section connected to the upper teeth fitting section with a plurality of connecting bridges, the plurality of connecting bridges fixing the position of the lower teeth fitting section relative to the upper teeth fitting section; and
airway passages formed between the connecting bridges, wherein
the lower teeth fitting section is adapted to position the patient's lower jaw to reduce or prevent sleep apnea, wherein the position is determined by a sleep disorder examination of the patient.

12. The method of claim 11, wherein the upper and lower teeth fitting sections contain at least 80% of the patient's teeth therewithin.

13. The method of claim 11, wherein a soft material is inserted into the upper and lower teeth fitting sections to cushion the teeth inside the oral appliance when worn by the patient.

14. The method of claim 11, wherein the upper and lower teeth fitting sections are formed from a hard material.

15. The method of claim 11, further comprising at least four connecting bridges and at least three airway passages formed between adjacent ones of the connecting bridges.

16. The method of claim 11, wherein the step of fitting the patient with the oral appliance includes conducting a sleep disorder examination on the patient.

17. The method of claim 11, wherein the sleep disorder examination includes three-dimensional radiological and clinical examinations.

18. The method of claim 11, wherein the sleep disorder examination includes taking the user's vital data, an airway evaluation, mallampati classification, tonsil classification, and a dental screening.

19. The method of claim 11, further comprising inserting a soft material is into the upper and lower teeth fitting sections to cushion the teeth inside the oral appliance when worn by the user.

20. The method of claim 11, further comprising clasping at least one of the user's molar or premolar teeth to aid in retention of the appliance.
